(19) United States
(12) Patent Application Publication
(10) Pub. No.: US 2006/0205733 A1
(43) Pub. Date: Sep. 14, 2006

(54) Endothelin a receptor antagonists in combination with phosphodiesterase 5 inhibitors and uses thereof

(75) Inventors: Richard Dixon, Houston, TX (US); Donald J. Keyser, South Lake, TX (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Encysive Pharmaceuticals, Houston, TX (US)

(21) Appl. No.: 11/211,099

(22) Filed: Aug. 25, 2005

Related U.S. Application Data
(60) Provisional application No. 60/604,462, filed on Aug. 26, 2004.

(51) Int. Cl. A61K 31/519; A61K 31/513; A61K 31/4745; A61K 31/42; A61K 31/4025

(52) U.S. Cl. 514/252.16; 514/262.1; 514/309; 514/291; 514/269; 514/422; 514/378

(57) ABSTRACT

The invention relates generally to combination therapies comprising an endothelin A receptor (ET_{A}) antagonist and a phosphodiesterase 5 (PDE5) inhibitor, pharmaceutical compositions comprising ET_{A} antagonist and PDE5 inhibitor and methods of treating various disorders comprising administering an ET_{A} antagonist and a PDE5 inhibitor. In particular, the combination therapies and pharmaceutical compositions are useful for the treatment and/or prevention of cardiac disorders such as pulmonary arterial hypertension (PAH).

### CROSS-REFERENCE TO RELATED APPLICATION

This application claims the benefit of U.S. Provisional Application No. 60/604,462, filed Aug. 26, 2004, which is hereby incorporated by reference in its entirety.

### FIELD OF THE INVENTION

The invention relates generally to combination therapies comprising an endothelin A receptor (ET_{A}) antagonist and a phosphodiesterase 5 (PDE5) inhibitor, pharmaceutical compositions comprising ET_{A} antagonist and PDE5 inhibitor and methods of treating various disorders comprising administering an ET_{A} antagonist and a PDE5 inhibitor. In particular, the combination therapies and pharmaceutical compositions are useful for the treatment and/or prevention of cardiac disorders such as pulmonary arterial hypertension (PAH).

### BACKGROUND OF THE INVENTION

Systemic hypertension, also called high blood pressure, is a condition in which the blood pressure in either arteries or veins is abnormally high. Blood pressure is defined as the force exerted by the blood against the walls of the blood vessels. Normally, the pumping of the heart creates a rhythmic pulsing of blood along and against the walls of the blood vessels, which are flexible enough to dilate or contract and thus keep the pressure constant. Most physicians consider the normal systemic blood pressure of a healthy adult to be approximately 120/80—i.e., equivalent to the pressure exerted by a column of mercury 120 mm high during contraction of the heart (systole) and 80 mm high during relaxation (diastole). However, for a variety of reasons, the blood vessels may lose their flexibility, or the muscles surrounding them may force them to contract. As a result, the heart must pump more forcefully to move the same amount of blood through the narrowed vessels into the capillaries, thereby increasing the blood pressure. Regardless of the mechanism, a sustained elevation of blood pressure for a period of time has been shown to result in significant cardiovascular damage throughout the body, e.g., congestive heart failure, coronary artery disease, stroke and progressive renal failure. Congestive heart failure frequently constitutes an end-stage complication of cardiac overload due to systemic hypertension or cardiac valve dysfunctions but may also result from acute or chronic ischemic heart disease and idiopathic cardiomyopathies (Battegay, J. Mol. Med., 73:333 (1995)). Patients suffering from systemic hypertension or aortic valve dysfunction can benefit from adequate drug treatment or valve replacements, but hypertrophy and heart failure may become irreversible (Golia et al., Int. J. Cardiol., 60:81 (1997)).

Systemic hypertension is generally classified by cause, either as essential (of unknown origin) or as secondary (the result of a specific disease, disorder or other condition). Secondary hypertension may result from a wide range of causes. For example, renal hypertension affects the entire systemic circulation and arises from hypertension within the renal arteries, which branch from the aorta to supply blood to the kidneys. Hypertension may also result from the excess hormones that are secreted during abnormal functioning of the outer substance, or cortex, of the adrenal glands (Cushing's syndrome; aldosteronism); from the excess hormones resulting from pheochromocytoma, which is a tumor of the inner substance (medulla) of the adrenal glands; or from the excess hormones secreted by pituitary tumors. Other causes of secondary hypertension are coarctation—localized narrowing—of the aorta, pregnancy, and the use of oral contraceptives. In all secondary cases, the hypertension is relieved by treating the underlying condition or cause. By far the most common form of hypertension (90 percent of cases) is essential, or idiopathic, hypertension. Although no specific cause can be determined in such cases, studies have pointed out several contributing factors. Included among these are a family history of hypertension, obesity, high salt intake, smoking, and most importantly, emotional and physical stress.

In its milder forms, essential hypertension is usually treated with a self-help regimen that includes a no-salt diet and perhaps a weight-reducing diet, a decrease in or cessation of smoking, mild exercise, and the avoidance of or successfully coping with stressful situations. If a self-help program does not help lower the patient's blood pressure, the physician will usually prescribe diuretics or sympathetic-nerve blockers. The nerve blockers generally act by decreasing heart output and peripheral resistance to blood flow. Beta blockers are the most commonly used of these drugs and include metoprolol, nadolol, and propranolol. More severe hypertension often requires the use of drugs called vasodilators, which dilate the arteries, thus lowering blood pressure. Oral vasodilators, which include hydralazine and minoxidil, are often used in conjunction with a diuretic and a sympathetic nerve blocker to inhibit the body's natural tendency to increase fluid retention and increase blood flow in response to the arterial dilation. Severe and immediately life-threatening hypertension, either secondary or essential, is called malignant hypertension and usually requires hospitalization and acute medical care. Treatment includes the intravenous administration of vasodilators such as diazoxide.

Cyclic nucleotide second messengers (cAMP and cGMP) play a central role in signal transductions and regulation of physiologic responses, such as vasodilation. Their intracellular levels are controlled by the complex superfamily of cyclic nucleotide phosphodiesterase (PDE) enzymes. Inhibitors of PDE are agents that can either activate or suppress PDEs via allosteric interaction with the enzymes or binding to the active site of the enzymes. The PDE family includes at least 19 different genes and at least 11 PDE isozyme families, with over 50 isozymes having been identified thus far. The PDEs are distinguished by (a) substrate specificity, i.e., cGMP-specific, cAMP-specific or nonspecific PDEs, (b) tissue, cellular or even sub-cellular distribution, and (c) regulation by distinct allosteric activators or inhibitors. PDE inhibitors include both nonspecific PDE inhibitors and specific PDE inhibitors (those that inhibit a single type of phosphodiesterase with little, if any, effect on any other type of phosphodiesterase).

Pulmonary arterial hypertension (PAH) is a condition that involves high blood pressure and structural changes in the walls of the pulmonary arteries, which are the blood vessels that connect the right side of the heart to the lungs. PAH causes shortness of breath, limits activity, and is eventually fatal unless treated successfully with heart and lung transplant. Primary and secondary PAH are estimated to afflict approximately 80,000 to 100,000 people worldwide, many of whom are children and young women.

Standard management of patients with PAH includes anticoagulant therapy with warfarin (COUMADIN, and others) in combination with a diuretic such as furosemide (LASIX, and others) to manage fluid retention caused by right-sided heart failure, and for selected patients, a calcium-channel blocker such as amlodipine (NORVASC) (J R Runo and J E Loyd, Lancet, 361:1533 (2003); J P Maloney, Curr. Opin. Pulm. Med.,; 9:139 (2003)). One phosphodiesterase type 5 (PDE5) inhibitor, sildenafil (REVATIO), has recently been approved for treating PAH in 20 mg doses (TID). PDE5 is the main phosphodiesterase in the pulmonary vasculature; inhibiting it maintains high levels of cGMP, which promotes the vasodilating effects of endogenous nitric oxide (M Humbert and G Simonneau, Am. J. Respir. Crit. Care Med., 169:6 (2004)).

However, PDE5s, such as sildenafil have several adverse effects. For instance, in intermittent use of sildenafil (VIAGRA) for erectile dysfunction, once-daily doses of 25-100 mg has caused headaches, dyspepsia and visual disturbances. Its most serious effect has been severe, sometimes fatal, hypotension in patients taking nitrates for angina pectoris (see, Abramowicz, ed., Sildenafil for Pulmonary Hypertension, The Medical Letter on Drugs and Therapeutics, Vol. 46, Issue 1177, (Mar. 1, 2004) Therefore, it would be beneficial to provide a complimentary treatment and reduce dosage amounts and/or side effects related to treatment with PDE5 inhibitors.

Endothelin is a peptide which is composed of 21 amino acids and is synthesized and released by the vascular endothelium. Endothelin exists in three isoforms: ET-1, ET-2 and ET-3. Endothelin is a potent vasoconstrictor and has a potent effect on vessel tone. The vasoconstricting effect is caused by the binding of endothelin to its receptor on the vascular smooth muscle cells (Nature, 332:411-415 (1988); FEBS Letters, 231:440-444 (1988); Biochem. Biophys. Res. Commun. 154:868-875 (1988)).

Increased or abnormal release of endothelin causes persistent vasoconstriction in the peripheral, renal and cerebral blood vessels, which may lead to illnesses. It has been reported in the literature that elevated levels of endothelin were found in the plasma of patients with hypertension, acute myocardial infarction, pulmonary hypertension, Raynaud's syndrome and atherosclerosis and in the airways of asthmatics (Japan J. Hypertension, 12:79 (1989); J. Vascular Med. Biology 2:207 (1990); J. Am. Med. Association 264:2868 (1990)).

Two distinct endothelin receptors, designated ET_{A} and ET_{B}, have been identified, and DNA clones encoding each receptor have been isolated (Arai et al., Nature, 348(6303):730-732 (1990); Sakurai et al., Nature, 348(6303):732-735 (1990)). Based on the amino acid sequences of the proteins encoded by the cloned DNA, it appears that each receptor contains seven membrane-spanning domains and exhibits structural similarity to G-protein-coupled membrane proteins. Messenger RNA encoding both receptors has been detected in a variety of tissues, including heart, lung, kidney and brain. The distribution of receptor subtypes is tissue specific (Martin et al., Biochem. Biophys. Res. Commun., 162:130-137 (1989)). ET_{A} appears to be selective for endothelin-1 and is predominant in cardiovascular tissues. ET_{B} is predominant in noncardiovascular tissues, such as the central nervous system and kidney, and interact with the three endothelin isopeptides (Sakurai et al., Nature, 348(6303):732-735(1990)). In addition, ET_{A} occurs on vascular smooth muscle, is linked to vasoconstriction and has been associated with cardiovascular, renal and central nervous system diseases whereas ET_{B} is located on the vascular endothelium, is linked to vasodilation (Takayanagi et al., FEBS Letters., 282:103-106 (1991)) and has been associated with bronchoconstrictive disorders.

By virtue of the distribution of receptor types and the differential affinity of each isopeptide for each receptor type, the activity of the endothelin isopeptides varies in different tissues. For example, endothelin-1 inhibits ¹²⁵I-labeled endothelin-1 binding in cardiovascular tissues forty to seven hundred times more potently than endothelin-3. ¹²⁵I-labeled endothelin-1 binding in non-cardiovascular tissues, such as kidney, adrenal gland, and cerebellum, is inhibited to the same extent by endothelin-1 and endothelin-3, which indicates that ET_{A} predominates in cardiovascular tissues and ET_{B} predominates in non-cardiovascular tissues.

Endothelin plasma levels are elevated in certain disease states (see, e.g., International Application No. WO 94/27979 and U.S. Pat. No. 5,382,569). Endothelin-1 plasma levels in healthy individuals, as measured by radioimmunoassay (RIA), are about 0.26-5 pg/ml. Blood levels of endothelin-1 and its precursor, big endothelin, are elevated in shock, myocardial infarction, vasospastic angina, kidney failure and a variety of connective tissue disorders. In patients undergoing hemodialysis or kidney transplantation or suffering from cardiogenic shock, myocardial infarction or pulmonary hypertension, blood levels of endothelin-1 as high as 35 pg/ml have been observed (see, Stewart et al., Annals Internal Med., 114:464-469 (1991)). Because endothelin is likely to be a local, rather than a systemic, regulating factor, it is probable that the levels of endothelin at the endothelium/smooth muscle interface are much higher than circulating levels.

Elevated levels of endothelin have also been measured in patients suffering from ischemic heart disease (Yasuda et al., Amer. Heart J., 119:801-806 (1990); Ray et al., Br. Heart J,. 67:383-386 (1992)). Circulating and tissue endothelin immunoreactivity is increased more than two-fold in patients with advanced atherosclerosis (Lerman et al., New Engl. J. Med., 325:997-1001 (1991)). Increased endothelin immunoreactivity has also been associated with Buerger's disease (Kanno et al., J. Amer. Med. Assoc., 264:2868 (1990)) and Raynaud's phenomenon (Zamora et al., Lancet, 336:1144-1147 (1990)). Increased circulating endothelin levels were observed in patients who underwent percutaneous transluminal coronary angioplasty (PTCA) (Tahara et al., Metab. Clin. Exp., 40:1235-1237 (1991); Sanjay et al., Circulation, 84(Suppl. 4):726 (1991)) and in individuals with pulmonary hypertension (Miyauchi et al., Jpn. J. Pharmacol., 58:279P (1992); Stewart et al., Ann. Internal Medicine, 114:464-469(1991)).

A recent study in patients with congestive heart failure demonstrated a good correlation between the elevated levels of endothelin in the plasma and the severity of the disease.

Endothelin is an endogenous substance that directly or indirectly (through the controlled release of various other endogenous substances) induces sustained contraction of vascular or non-vascular smooth muscles. Its excess production or excess secretion is believed to be one of the factors responsible for hypertension, pulmonary hypertension, Raynaud's disease, bronchial asthma, acute renal failure, myocardial infarction, angina pectoris, arteriosclerosis, cerebral vasospasm and cerebral infarction (see A. M. Doherty, Endothelin: A New Challenge., J. Med. Chem., 35:1493-1508 (1992)).

Substances that specifically inhibit the binding of endothelin to its receptor are believed to block the physiological effects of endothelin and are useful in treating patients with endothelin-related disorders.

### SUMMARY OF THE DISCLOSURE

One embodiment of the invention is directed to a combination therapy comprising at least one endothelin A receptor (ET_{A}) antagonist and a phosphodiesterase 5 (PDE5) inhibitor.

Another embodiment of the invention is directed to a combination therapy, wherein the ET_{A} antagonist and the PDE5 inhibitor are administered together or separately

Another embodiment of the invention provides for the combination therapy to be a pharmaceutical composition, wherein the pharmaceutical composition is in an immediate release formulation or a controlled release formulation, wherein the ET_{A} antagonist is in a controlled release formulation, the PDE5 inhibitor is in a controlled release formulation or both are in a controlled release formulation. If both are in a controlled release formulation, the ET_{A} antagonist and the PDE5 inhibitor may be released at different rates.

Another embodiment of the invention is directed to a pharmaceutical composition comprising endothelin A receptor (ET_{A}) antagonist, a phosphodiesterase 5 (PDE5) inhibitor and a pharmaceutical carrier.

Another embodiment of the invention provides for the pharmaceutical composition to be in an immediate release formulation or a controlled release formulation, wherein the ET_{A} antagonist is in a controlled release formulation, the PDE5 inhibitor is in a controlled release formulation or both are in a controlled release formulation. If both are in a controlled release formulation, the ET_{A} antagonist and the PDE5 inhibitor may be released at different rates.

Yet another embodiment provides for a method of reducing the side effects or toxicity of an ET_{A} antagonist, a PDE5 inhibitor or both comprising administering an ET_{A} antagonist and a PDE5 inhibitor, wherein the amount of the PDE5 required to treat a condition is reduced or modulated.

Another embodiment of the invention is directed to a method of treating pulmonary hypertension comprising administering to a subject in need thereof an effective amount of a) a PDE5 inhibitor and b) an ET_{A} antagonist, wherein administration of a) and b) is concurrent or sequentially in either order.

Another embodiment of the invention is directed to a method of effecting or facilitating the treatment of a vascular condition in a mammal comprising administering a therapeutically effective amount of an ET_{A} antagonist and a PDE5 inhibitor.

Another embodiment of the invention is directed to a method of treating pulmonary arterial hypertension comprising administering to a subject in need thereof a therapeutically effective amount of an ET_{A} antagonist and a PDE5 inhibitor.

Another embodiment of the invention is directed to a method for treating a vascular condition comprising administering to a subject in need thereof a therapeutically effective amount of a combination of ET_{A} antagonist and a PDE5 inhibitor.

For each of the recited embodiments described in the application, the ET_{A} antagonist may be selected from any one of the compounds described herein selective for the endothelin A receptor or as can be determined according to references cited herein, and the PDE₅ inhibitor may be selected from any one of the compounds described herein as selective for PDE5 or as can be determined according to references cited herein. One example for each of the recited embodiments comprises sitaxsentan as the ET_{A} antagonist and sildenafil, tadalafil (CIALIS), vardenafil (LEVITRA) or dasantafil as the PDE5 inhibitor. In another example for each of the recited embodiments described in the application, the combination comprises sitaxsentan and sildenafil. In yet another example for each of the recited embodiments described in the application, the combination comprises sitaxsentan and tadalafil.

Embodiments of the invention are useful in treating vascular conditions such as erectile dysfunction, atherosclerosis, renal failure, hypertension, congestive heart failure, diabetic nephropathy, diabetic neuropathy, interstitial lung disease, obstructive sleep dyspnea, obstructive sleep apnea and resistant hypertension.

Embodiments of the invention are also useful in treating cardiovascular disorders such as hypertension, pulmonary hypertension, postischemic renal failure, vasospasm, cerebral and cardiac ischemia, myocardial infarction, endotoxic shock, benign prostatic hyperplasia, complications of diabetes, migraine, bone resorption and inflammatory diseases, including Raynaud's disease and asthma. In one embodiment, the condition treated according to the invention is pulmonary arterial hypertension.

Methods of the invention include dual drug tablets comprising both an ET_{A} antagonist and a PDE5 inhibitor. However, the two drugs may be provided in separate dosage formulations so that each may be administered substantially concurrently or at sequentially to provide required therapeutic amounts.

Improvement among a subject group may be measured through any number of ways known by those of ordinary skill in the art.

The present invention will now be described in more detail with reference to exemplary embodiments thereof as shown in the accompanying drawings. While the present invention is described below with reference to exemplary embodiments, it should be understood that the present invention is not limited thereto. Those of ordinary skill in the art will recognize additional implementations, modifications and embodiments that are within the scope of the present invention, as well as other fields of use that may be of significance, in view of the present disclosure.

### BRIEF DESCRIPTION OF THE DRAWINGS

In order to facilitate a fuller understanding of the present invention, reference is now made to the accompanying drawings. These drawings should not be construed as limiting the present invention, but are intended to be exemplary only.

FIG. 1 is a depiction of ABT-627 (artrasentan), an ET_{A}-selective inhibitor.

FIG. 2 is a depiction of ABT-546, an ET_{A}-selective inhibitor.

FIG. 3 depicts sitaxsentan.

FIG. 4 depicts N-Oxazole thiophene sulfonamides described in Wu, et al., Recently discovered sulfonamide-, acyl sulfonamide- and carboxylic acid-based endothelin antagonists, Drugs, 6(3):232-239 (2003).

FIG. 5 depicts mean plasma concentrations of sildenafil after oral administration of a single 100 mg dose of sildenafil to healthy subjects following 100 mg doses of sitaxsentan or placebo daily for seven days.

FIG. 6 depicts mean plasma concentrations of N-desmethyl sildenafil after oral administration of a single 100 mg dose of sildenafil to healthy subjects following 100 mg doses of sitaxsentan or placebo daily for seven days.

### DETAILED DESCRIPTION OF EXEMPLARY EMBODIMENTS

All publications, patents and patent applications cited in this specification are hereby incorporated by reference as if each individual publication, patent or patent application were specifically and individually indicated to be incorporated by reference. Although the present invention is described in some detail by way of illustrations and examples for purposes of clarity of understanding, it will be readily apparent to those of ordinary skill in the art that certain changes and modifications may be made thereto without departing from the spirit or scope of the appended claims in view of the teachings of the present invention.

“Pulmonary hypertension” is a specific condition of hypertension in the lung and relates to arterial hypertension, capillary hypertension or venous hypertension in the lung. The term “pulmonary hypertension” relates to pulmonary arterial hypertension (PAH). Furthermore it will be understood that pulmonary arterial hypertension relates to, but is not restricted to, both primary arterial hypertension and to pulmonary arterial hypertension occurring secondary to pulmonary diseases such as chronic bronchitis, emphysema, kyphoscoliosis and conditions such as chronic mountain sickness. Pulmonary hypertension is a serious medical condition that may lead to right ventricular hypertrophy, failure and death. When used herein the term “right heart failure” relates to disorders such as cor pulmonale and congenital abnormalities of the heart. It will be appreciated that cor pulmonale often occurs secondary to certain lung diseases such as chronic bronchitis and emphysema. Congenital abnormalities of the heart include disorders such as atrial septal defect, tetralogy of fallot, venticular septal defect and persistent ductus arteriosus.

Phosphodiesterase Inhibitors

One phosphodiesterase type 5 (PDE5) inhibitor, sildenafil (REVATIO), has recently been approved for treating PAH in 20 mg doses (TID). PDE5 is the main phosphodiesterase in the pulmonary vasculature; inhibiting it maintains high levels of cGMP, which promotes the vasodilating effects of endogenous nitric oxide (M Humbert and G Simonneau, Am. J. Respir. Crit. Care Med., 169:6 (2004)).

However, PDE5s, such as sildenafil, have several adverse effects. For instance, in intermittent use of sildenafil (VIAGRA) for erectile dysfunction, once-daily doses of 25-100 mg has caused headaches, dyspepsia and visual disturbances. Its most serious effect has been severe, sometimes fatal, hypotension in patients taking nitrates for angina pectoris (see, Abramowicz, ed., Sildenafil for Pulmonary Hypertension, The Medical Letter on Drugs and Therapeutics, Vol. 46, Issue 1177, (Mar. 1, 2004). The side effects are also present when using REVATIO to treat PAH. Therefore, it would be beneficial to combine a PDE5 inhibitor with an ET_{A} antagonist to provide complimentary treatment and reduce dosage amounts and/or side effects related to treatment with PDE5 inhibitors.

For each of the recited embodiments, useful phosphodiesterase type 5 inhibitors include, e.g., vardenafil (LEVITRA), tadalafil (CIALIS), zaprinast, MBCQ, MY-5445, dipyridamole, furoyl and benzofuroyl pyrroloquinolones, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride (T-0156) and T-1032 (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), and sildenafil. PDE5 inhibitors which may be mentioned by way of example are RX-RA-69, SCH-51866, KT-734, vesnarinone, zaprinast, SKF-96231, ER-21355, BF/GP-385 and NM-702. Additional PDE5 inhibitors and their structures are described, for instance, in U.S. Pat. No. 5,250,534 and U.S. Pat. No. 6,469,012. Other forms of the PDE5 inhibitor, such as isomers (e.g. resolved enantiomers or racemic mixtures), metabolites, polymorphs, salts and complexes thereof, may also be used in an embodiment of the invention.

In one embodiment, mixtures of the aforementioned PDE5 inhibitors is used. In another embodiment, the PDE5 inhibitor is tadalafil. In yet another embodiment, the PDE5 inhibitor is sildenafil. Sildenafil citrate is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate and has the following structural formula: 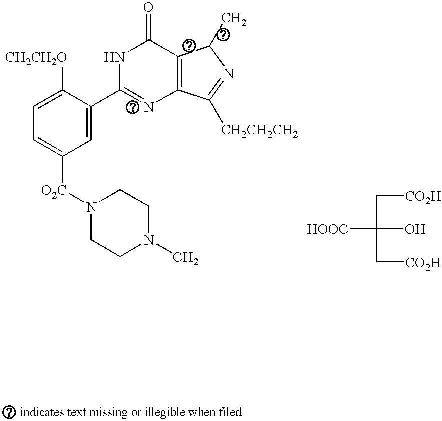
Endothelin Receptor Antagonists

Because endothelin is associated with certain disease states and is implicated in numerous physiological effects, compounds that can interfere with or hinder endothelin-associated activities, such as endothelin-receptor interaction and vasoconstrictor activity, are of interest. Several compounds that are endothelin receptor antagonists have been identified. For example, a fermentation product of Streptomyces misakiensis, designated BE-18257B, has been identified as an ET_{A} antagonist. BE-18257B is a cyclic pentapeptide (cyclo(D-Glu-L-Ala-allo-D-Ile-L-Leu-D-Trp)), which inhibits ¹²⁵I-labeled endothelin-1 binding in cardiovascular tissues in a concentration-dependent manner (IC₅₀ 1.4 μM in aortic smooth muscle, 0.8 μM in ventricle membranes and 0.5 μM in cultured aortic smooth muscle cells) but fails to inhibit binding to receptors in tissues in which ET_{B} predominates at concentrations up to 100 μM. Cyclic pentapeptides related to BE-18257B, such as BQ-123 (cyclo(D-Asp-Pro-D-Val-Leu-D-Trp)), have been synthesized and have also been shown to be ET_{A} antagonists (see, U.S. Pat. No. 5,114,918 to Ishikawa et al.; see, also, EP A1 0 436 189 to Banyu Pharmaceutical Co., Ltd (Oct. 7, 1991)). Studies that measure the inhibition by these cyclic peptides of endothelin-1 binding to endothelin-specific receptors indicate that these cyclic peptides bind preferentially to ET_{A}. Other peptide and non-peptidic ET_{A} antagonists have been identified (see, e.g., U.S. Pat. Nos. 5,352,800; 5,334,598; 5,352,659; 5,248,807; 5,240,910; 5,198,548; 5,187,195 and 5,082,838). These include other cyclic peptides, acyltripeptides, hexapeptide analogs, certain anthraquinone derivatives, indanecarboxylic acids, certain N-pyrimidinylbenzenesulfonamides, certain benzenesulfonamides, and certain naphthalenesulfonamides (Nakajima et al., J. Antibiot., 44:1348-1356 (1991); Miyata et al., J. Antibiot., 45:74-78(1992); Ishikawa et al., J. Med. Chem., 35:2139-2142 (1992); U.S. Pat. No. 5,114,918 to Ishikawa et al.; EP A1 0 569 193; EP A1 0 558 258; EP A1 0 436 189 to Banyu Pharmaceutical Co., Ltd (Oct. 7, 1991); Canadian Patent Application No. 2,067,288; Canadian Patent Application No. 2,071,193; U.S. Pat. No. 5,208,243; U.S. Pat. No. 5,270,313; Cody et al., Med. Chem. Res., 3:154-162 (1993); Miyata et al., J. Antibiot., 45:1041-1046 (1992); Miyata et al., J. Antibiot., 45:1029-1040 (1992); Fujimoto et al., FEBS Letters, 305:41-44 (1992); Oshashi et al., J. Antibiot., 45:1684-1685 (2002); EP A1 0 496 452; Clozel et al., Nature, 365:759-761 (1993); International Patent Application No. WO 93/08799; Nishikibe et al., Life Sci., 52:717-724 (1993); and Benigni et al., Kidney Int., 44:440-444 (1993)). In general, the identified compounds have ET_{A} antagonist activity in in vitro assays at concentrations on the order of about 50-100 mM or less. A number of such compounds have also been shown to possess activity in in vivo animal models.

It has been recognized that compounds that exhibit activity at IC₅₀ or EC₅₀ concentrations on the order of 10⁻⁴ mM or lower in standard in vitro assays that assess endothelin antagonist or agonist activity have pharmacological utility (see, e.g., U.S. Pat. Nos. 5,352,800; 5,334,598; 5,352,659; 5,248,807; 5,240,910; 5,198,548; 5,187,195 and 5,082,838). By virtue of this activity, such compounds are considered to be useful for the treatment of hypertension such as peripheral circulatory failure, heart disease such as angina pectoris, cardiomyopathy, arteriosclerosis, myocardial infarction, pulmonary hypertension, vasospasm, vascular restenosis, Raynaud's disease, cerebral stroke such as cerebral arterial spasm, cerebral ischemia, late phase cerebral spasm after subarachnoid hemorrhage, asthma, bronchoconstriction, renal failure, particularly post-ischemic renal failure, cyclosporine nephrotoxicity such as acute renal failure, colitis, as well as other inflammatory diseases, endotoxic shock caused by or associated with endothelin and other diseases in which endothelin has been implicated.

Therefore, compounds showing endothelin receptor antagonistic activity have prophylactic and therapeutic effects against diseases caused by ischemia, for example, cerebral infarction, angina pectoris, myocardial infarction and renal insufficiency.

Thus, in view of the association of endothelin with numerous diseases, endothelin is believed to play a critical role in these pathophysiological conditions (see, Saito et al., Hypertension 15:734-738 (1990); Tomita et al., N. Engl. J. Med., 321: 1127 (1989); Kurihara et al., J. Cardiovasc. Pharmacol. 13(Suppl. 5):S13-S17 (1989); Doherty, J. Med. Chem. 35:1493-1508 (1992); Morel et al., Eur. J. Pharmacol., 167:427-428 (1989)).

Accordingly, substances that specifically inhibit the binding of endothelin to its receptor (i.e. antagonists) should prevent the various above-mentioned physiological effects of endothelin and therefore, be valuable drugs. For example, endothelin receptor antagonists of the present invention can be used for the treatment of hypertension, pulmonary hypertension, myocardial infarction, angina pectoris, acute kidney failure, renal insufficiency, cerebral vasospasms, cerebral ischemia, subarachnoid hemorrhages, migraine, asthma, atherosclerosis, endotoxic shock, endotoxin-induced organ failure, intravascular coagulation, restenosis after angioplasty, benign prostate hyperplasia, hypertension or kidney failure caused by ischemia or intoxication as described in International Application Nos. WO96/11914 and WO95/26716.

Two types of mammalian endothelin (ET) receptors, ET_{A} and ET_{B}, have been characterized. ET_{A} is selective for ET-1 and ET-2, while ET_{B} binds ET-1, ET-2 and ET-3 with equal affinity. ET_{A} mediates vasoconstriction and cell proliferation, whereas ET_{B} is important for the clearance of ET-1, endothelial cell survival, the release of nitric oxide and prostacyclin, and the inhibition of ECE-1 (see, Luscher, T. et al., Endothelins and Endothelin Receptor Antagonists, Therapeutic Considerations for a Novel Class of Cardiovascular Drugs, Circulation, 2434-2444 (Nov. 7, 2000); Wu-Wong, et al, Pharmacology of endothelin receptor antagoinists ABT-627, ABT-546, A-182086 and A-192621: in vitro studies, Clinical Science, Suppl. 48, 107-111 (2002)).

A major advance was made in the ET field with the development of endothelin receptor antagoinists. BQ-123 and FR139317, two peptidic ET_{A}-selective antagonists, are important advancements in the investigation of ET-mediated pathophysiology. Following the peptidic compounds, a number of nonpeptide antagonists with improved pharmacokinetics, such as Ro 47-0203, SB 217242, atrasentan, etc., were developed.

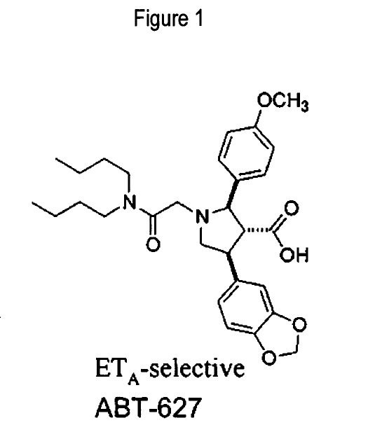

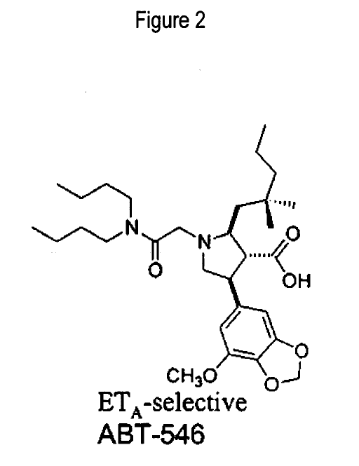

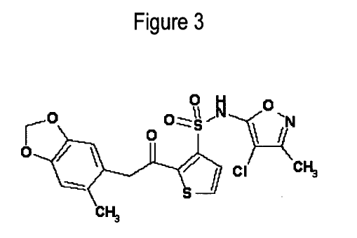

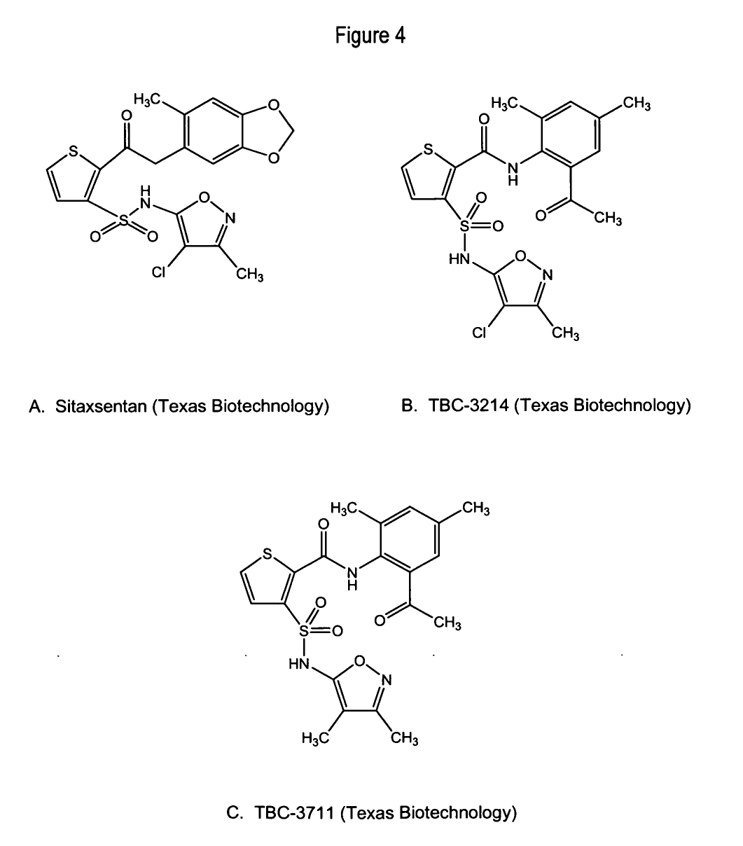

Examples of endothelin receptor antagonists include, but are not limited to, BE 1827, BQ-610, ABT 627 (see FIG. 1), ABT 546 (see FIG. 2), Ro 61-1790, ZD1611, BMS-182874, BMS-193884, sitaxsentan (TBC 11251) (see FIG. 3), EMD 122946, J-104132, LU 127043, LU 135252, SB 234551, SB 247083, and their derivatives, etc. (see, Doherty, Annual Reports in Medicinal Chemistry, 35:73-82 (Academic Press, 2000)). Other ethenesulfonamide derivatives, which are endothelin receptor antagonists and are useful in the methods of the present invention, are disclosed by Harada, et al., Chem. Pharm. Bull., 49(12):1593-1603 (2001). N-Oxazole thiophene sulfonamides (see FIG. 4) are described in Wu, et al., Recently discovered sulfonamide-, acyl sulfonamide- and carboxylic acid-based endothelin antagonists, Drugs, 6(3):232-239 (2003) Other ET_{A} antagonists are described, for instance, in U.S. Patent Application Publication Nos. 20030092757 and 20030040534. Other forms of the ET_{A} antagonist, such as isomers (e.g. resolved enantiomers or racemic mixtures), metabolites, polymorphs, salts and complexes thereof, may also be used in an embodiment of the invention.

In one embodiment, the ET_{A} antagonist is sitaxsentan, which is commercially marketed under the trademark THELIN. U.S. Pat. Nos. 5,591,761; 5,594,021; 5,962,490; 6,248,767 and 6,458,805 disclose compositions including sitaxsentan, methods of using sitaxsentan and pharmaceutical compositions comprising sitaxsentan. U.S. Pat. No. 5,783,705 discloses methods of making sitaxsentan.

THELIN (sitaxsentan sodium; TBC11251Na) is an orally active endothelin A receptor antagonist that has been developed to treat pulmonary arterial hypertension (PAH). ET-1, produced primarily by vascular endothelial cells, is the predominant isoform found within the cardiovascular system and is a potent endogenous vasoconstrictor with proliferative and profibrotic effects. ET-1 also influences salt and water homeostasis as well as the renin-angiotensin-aldosterone and sympathetic nervous systems. There is a substantial body of experimental work suggesting that the primary physiologic effects of ET-1 in experimental animals and patients with PAH are sustained vasoconstriction of the pulmonary vasculature with remodeling due to proliferation or hypertrophy of vascular smooth muscle.

Within the cardiovascular system, the effects of ET-1 on vascular smooth muscle cells and cardiac myocytes are believed to be principally mediated through the ET_{A}. Activation of ET_{A} facilitates sustained vasoconstriction of vascular smooth muscle, stimulation of, proliferation of, and hypertrophy of vascular smooth muscle cells, positive inotropic activity, and hypertrophy of cardiac cells. In contrast, ET_{B} is found primarily on endothelial cells, kidney, and central nervous tissue and are involved in the clearance of ET-1, particularly in the vascular beds of the lung and kidney. Endothelial ET_{B} facilitates vasodilation due to the release of smooth muscle relaxants such as nitric oxide and prostacyclin. Important stimuli for the release of ET-1 include, but are not limited to, hypoxia, ischemia, catecholamines, and angiotensin II. THELIN has a high specificity for ET_{A}, being approximately 6,500-fold more selective as an antagonist for ET_{A} compared to ET_{B}.

Selective compounds for ET_{A} antagonists according to this invention, in general, should display a relative receptor binding ratio of at least 100, such as more than 1000, such as in increments of 500 greater than 1000, i.e. 1500, 2000, 2500, etc., if they are going to act at only one of the receptor subtypes, e.g. ET_{A}.

Based upon the in vitro receptor affinity ratios, bosentan, which is the only endothelin receptor antagonist approved for the treatment of PAH, has an ET_{A}:ET_{B} selectivity ratio of 20 and is classified as a nonselective antagonist. In contrast, sitaxsentan has an ET_{A}:ET_{B} selectivity ratio of 6500 and is classified as an ET_{A}-selective antagonist. Therefore, for the purposes of this invention, bosentan or any other non-specific endothelin receptor antagonist would not represent an ET_{A}-specific antagonist.

THELIN is a small molecule that blocks the action of endothelin, a potent mediator of blood vessel constriction and growth of smooth muscle in vascular walls. Endothelin receptor antagonists are effective in the treatment of a variety of diseases where the regulation of vascular constriction is important. Side effects of THELIN include liver dysfunction (increased ALT and AST), headache, edema, constipation, nasal congestion and flushing.

Since ET_{A} appears to be selective for endothelin-1, examples of compounds that may be useful include, for instance, compounds described in U.S. Pat. No. 5,686,478 and Table 1.

TABLE 1
Endothelin Antagonists

| Compound | Target | Company | Indication/Comments |
|---|---|---|---|
| 12m | ET_{A} | Rhone-Poulenc Rorer | Cardiovascular diseases |
| A-127772 | ET_{A} | Abbott | |
| ABT-627 | ET_{A} | Abbott | CHF; prostate cancer |
| BE-18572A/B | ET_{A} | Banyu | |
| BMS-20794 | ET_{A} | Bristol Myers Squibb | |
| BMS-182874 | ET_{A} | Bristol Myers Squibb | CHF |
| BMS-193884 | ET_{A} | Bristol Myers Squibb | |
| BQ-123 | ET_{A} | Banyu | Intravenous use only |
| BQ-153 | ET_{A} | Banyu | Intravenous use only |
| BQ-162 | ET_{A} | Banyu | Intravenous use only |
| BQ-485 | ET_{A} | Banyu | Intravenous use only |
| BQ-610 | ET_{A} | Banyu | Intravenous use only |
| EMD-122946 | ET_{A} | Merck | CHF; hypertension |
| EMD-94246 | ET_{A} | Merck | CHF; hypertension |
| FR-139317 | ET_{A} | Fujisawa Pharm. Co. | |
| J-104121 | ET_{A} | Merck/Banyu | |
| J-104132 | ET_{A} | Merck/Banyu | Hypertension |
| L-744453 | ET_{A} | Merck | Cardiovascular diseases |
| L-749329 | ET_{A} | Merck | |
| L-754142 | ET_{A} | Merck | |
| LU127043 | ET_{A} | Knoll | |
| LU135252 | ET_{A} | Knoll | CHF; hypertension (darusentan) |
| LU208075 | ET_{A} | Knoll | CHF; hypertension |
| LU302146 | ET_{A} | Knoll | Occlusive vascular disease |
| PD-147953 | ET_{A} | Parke-Davis | Cardiovascular diseases |
| PD-151242 | ET_{A} | Parke-Davis | Cardiovascular diseases |
| PD-155080 | ET_{A} | Parke-Davis | Cardiovascular diseases |
| PD-156707 | ET_{A} | Parke-Davis | Cardiovascular diseases |
| RO 61-1790 | ET_{A} | Hoffmann-La Roche | SAH; intravenous use only |
| S-0139 | ET_{A} | Shionogi | Cardiovascular diseases |
| SB-234551 | ET_{A} | SmithKline Beecham | |
| SB-247083 | ET_{A} | SmithKline Beecham | |
| TA-0115 | ET_{A} | Tanabe Seiyaku Co., Ltd Heart | |
| | | failure | |
| TA-0201 | ET_{A} | Tanabe Seiyaku Co., Ltd Heart | |
| | | failure | |
| TBC11251 | ET_{A} | Texas Biotechnology Company | CHF; primary pulmonary |
| | | | hypertension |
| WS-7338B | ET_{A} | Fujisawa | |
| ZD 1611 | ET_{A} | Zeneca | Obstructive lung disease; |
| | | | primary pulmonary hypertension |

Adapted from Luscher et al., Endothelins and Endothelin Receptor Antagonists: Therapeutic Considerations for a Novel Class of Cardiovascular Drugs, 2434-2340 at http://www.circulationaha.org.

“ET_{A} antagonist” means any naturally occurring or synthetic compound that binds to the ET_{A} and blocks or inhibits the function of ET-1 or other agonist at that receptor. The ET_{A} antagonist may be a peptide or a non-peptide compound. Preferably, ET_{A} antagonists have a K_{d} for the ET_{A} of <1 μM, more preferably <100 nM, most preferably <10 nM, or even <1 nM. ET_{A} antagonists include, for example, sulfisoxazole, TBC-1 1251, BQ-123, BQ-610, BQ-745, PD 156707, PD 151242, TTA-386, JKC-301, JKC-302, BE-18257A, BE-18257B, A-1277722, LU 135252, TAK-044, SB 209670, SB 217242, FR139317, and ABT-627 (Table 2; Cheng et al., Ann. Reports in Medicinal Chemistry, Section II, Ch. 7, Endothelin Inhibitors, 61-70, (A. M. Doherty, ed., Academic Press, Inc. 1997).

TABLE 2
Binding K₁ (nM) to ET_{A} and ET_{B} for various ET receptor antagonists.

| Compound | Company | ET_{A} | ET_{B} | Selectivity | Reference |
|---|---|---|---|---|---|
| ABT-627 | Abbott | 0.034 | 63.8 | 1862 | [24] |
| ABT-546 | Abbott | 0.46 | 13,000 | 28,261 | [23] |
| BMS-182874 | Bristol-Myers Squibb | 48 | >50,000 | >1000 | [25] |
| FR-139317 | Fujisawa | 1.0 | 7300 | 7300 | [12] |
| J-104132 | Merck/Banyu | 0.034 | 0.1 | 2.9 | [26] |
| LU-135252 | BASF/Knoll | 1.4 | 184 | 130 | [27] |
| PD-156707 | Parke-Davis | 0.17 | 139 | 818 | [28] |
| Bosentan | Roche | 6.5 | 343 | 53 | [29] |
| Ro-61.1790 | Roche | 0.13 | >130 | 1000 | [30] |
| S-0139 | Shionogl | 1.0 | 1000 | 1000 | [31] |
| SB-209670 | SmithKline Beecham | 0.20 | 18 | 90 | [32] |
| SB-217242 | SmithKline Beecham | 1.1 | 111 | 101 | [33] |
| T-0201 | Tanabe | 0.015 | 41 | 2700 | [34] |
| TAK-044 | Takeda | 1.3 | 690 | 454 | [35] |
| TBC-11251 | Texas Biotechnology | 0.43 | >4300 | 10,000 | [36] |
| ZD-1811 | Zeneca | 1.3* | >2000 | 1500 | [37] |

*K₁ estimated from the IC_{∞} value (see, Wu-Wong, Endothelin Antagonists: Past, Present and Future, Current Opinion in Cardiovascular, Pulmonary & Renal Investigational Drugs, 1(3): 346-351 (1999)).

Some examples of ET receptor antagonists have undergone clinical development (see Table 3).

TABLE 3
ET receptor antagonists that have undergone clinical development.

| | | | Dev | |
|---|---|---|---|---|
| Compound | Selectivity | Administration | Phase | Indication |
| ABT-627 | ET_{A} | po | II | Prostate cancer |
| BMS-182874 | ET_{A} | iv | DX | CHF |
| BMS-193884 | ET_{A} | po | I | CHF, pulmonary |
| | | | | hypertension |
| FR-139317 | ET_{A} | iv | DX | Hypertension, CHF, |
| | | | | ischemia |
| J-104132/ | ET_{A}/ET_{A} | po | I | Hypertension, CHF |
| L-753037 | | | | |
| LU-135252 | ET_{A}/ET_{A} | po | II | CHF, other |
| | | | | cardiovascular |
| | | | | indications |
| Bosentan | ET_{A}/ET_{A} | po | III | CHF, hypertension, |
| | | | | ischemia |
| Ro-48-5695 | ET_{A} | po | II | CHF |
| Ro-61-0612 | ET_{A} | po, iv | I | CHF, other |
| | | | | cardiovascular |
| Ro-61-1790 | ET_{A} | iv | I | ARF, SH |
| S-0139 | ET_{A} | iv | I | Acute CHF, |
| | | | | hypertension, CI |
| SB-209670 | ET_{A}/ET_{A} | iv | II | ARF |
| SB-217242 | ET_{A}/ET_{A} | po | II | COPD, urological |
| TAK-044 | ET_{A}/ET_{A} | iv | II | MI, ARF, ACI, |
| | | | | hepatoprotective |
| TBC-11251 | ET_{A} | iv, po | IIa | CHF, COPD, |
| | | | | hypertension, SH |
| ZD-1611 | ET_{A} | po | I | CHF, pulmonary |
| | | | | hypertension |

ACI acute cerebral ischemia;
ARF acute renal failure;
COPD chronic obstructive pulmonary disease;
CHF congestive heart failure;
MI myocardial intarction;
SM subarachnoid hemorrhage;
DX discontinued

For a discussion of the role of endothelin in hypertension see Krum, H. et al., Role of endothelin in hypertension and therapeutic potential of endothelin blockade, Cardiovascular, Pulmonary & Renal Investigational Drugs, 1(3):316-329 (1999). Table 4 outlines several biochemical and pharmacological properties of sitaxentan in comparison to bosentan.

TABLE 4
Biochemical and pharmacological properties of sitaxsentan
in comparison to bosentan.

| Property | Sitaxsentan | Bosentan |
|---|---|---|
| Intrinsic potency to ET_{A} | Ki = 0.45 nM | Ki = 4.1 nM |
| t_{1/2} | 10 hours | 5 hours |
| Selectivity for ET_{A}:ET_{B} | 6500 | 20 |
| Effect on bile salt. | Does not accumulate | Inhibits bile salt |
| | bile salts. | export pump, results in |
| | | accumulation of bile |
| | | salts in hepatocytes. |
| Effect on bilirubin. | No effect. | Has been shown to |
| | | induce bilirubin |
| | | accumulation in PAH |
| | | patients. |
| Induction of cytochrome | No induction | Causes inhibition, |
| P450 enzymes. | demonstrated to date | followed by induction |
| | clinically. | of several CYP |
| | | enzymes, including |
| | | 3A4, 2C9, 2C19 |
| Metabolism | Through 3A4 and 2C9 | Through 3A4 and 2C9 |
| Route of elimination | Mixed Hepatic and | Hepatic |
| | Renal | |

For each of the recited embodiments, useful ET_{A} antagonists have been described above.

Combination Therapies

The principle drawback for using sildenafil in treating PAH is that it requires a high dose three times a day (much higher than the dose for erectile dysfunction, which is 15 mg to 75 mg periodically). Currently, the only endothelin receptor antagonist which has been approved for use in PAH is bosentan, which is a nonselective compound that blocks both the A and the B receptors. Use of a nonselective ET receptor interferes with multiple pathways whereas use of a specific ET_{A} antagonist will act in a complementary fashion for the multiple pathways (PDE and/or prostacyclin and/or ET_{A}) to provide superior efficacy and/or dosage regimes and/or reduction in side effects.

For instance, ET_{A} causes vasoconstriction, while ET_{B} causes vasodilatation. Bosentan works by blocking both ET_{A} and ET_{B} receptors. Sitaxsentan works by only blocking the ET_{A} and leaving the ET_{B} unimpaired. The mechanism by which ET_{B} causes vasodilatation is through stimulating the production of nitrous oxide and prostacylin. Nitrous oxide (NO) in turn activates the guanyl cyclase which increases the level of cGMP. The cGMP is responsible for relaxing the blood vessel. PDE5 acts to break down cGMP, so a PDE5 inhibitor also raises the level of cGMP, causing vasodilatation. Thus, when used together, increasing cGMP through the ET_{B} receptor and preventing its breakdown leads to increased vasodilatation and better efficacy of both drugs. Nonselective antagonists would not function as effectively because they block the ET_{B} stimulated cGMP production. Additionally in a recent study, Bosentan, a non-selective antagonist, was tested with sildenafil, but the study was terminated due to pharmacokinetic drug interaction problems.

Accordingly, the present invention relates to the use of a PDE5 inhibitor with an ET_{A}-specific antagonist therapy in inhibiting and preventing a cardiac stress or PAH. The methods and formulations of the invention provide new therapeutic approaches for the treatment and prevention of PAH in animals. “Treatment” or “treating” is also meant to encompass maintenance of cardiac conditions including PAH in a dormant (or quiescent) state at their primary site as well as secondary sites. Further, by “treating” or “treatment,” it is meant to increase the efficacy as well as prevent or decrease resistance to therapeutic modalities. “Treating” or “treatment” is also meant to encompass prevention of recurrence, reduction of pain, discomfort, and disability (morbidity), and an increase in quality of life associated with the condition. By “increasing the efficacy”, it is meant to include an increase in potency and/or activity of either the ET_{A} antagonist and/or the PDE5 inhibitor and/or a decrease in the required dosage. Status of patients receiving treatment may be evaluated by standards known in the art. For instance, a patient suffering from PAH may be subject to a 6 minute exercise walking test before and after the treatment period.

A “therapeutically effective dose” refers to that amount of the active agents that results in achieving the desired effect. Toxicity and therapeutic efficacy of such active agents can be determined by standard pharmaceutical procedures in cell cultures or in experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The data obtained from such data can be used in formulating a range of dosages for use in humans. The dosage of the active agents preferably lies within a range of circulating concentrations that include the ED₅₀ with little or no toxicity. The dosage can vary within this range depending upon the dosage form employed, and the route of administration utilized.

The exact formulation, route of administration, and dosage is determined by an individual physician in view of the patient's condition. Dosage amount and interval can be adjusted individually to provide levels of the active agents that are sufficient to maintain therapeutic or prophylactic effects.

The amount of pharmaceutical composition administered is dependent on the subject being treated, on the subject's weight, the severity of the affliction, the manner of administration, and the judgment of the prescribing physician. One embodiment of the invention contemplates the sequential administration of either the ET_{A} antagonist or the PDE5 inhibitor in separate daily dosages such that one may be given bi-daily while the other is provided once or three times a day. Likewise, sequentially is meant to include variations such as every other day treatment of agent and daily or multiple daily administrations of the other agent as required to achieve therapeutic effect. The combination therapy may be accomplished by providing the PDE5 inhibitor and the ET_{A} antagonist in separate dosage forms packaged together with instructions for the dosage administration regimen.

It is greatly preferred that the PDE5 inhibitor and ET_{A} antagonist or a pharmaceutically acceptable salt thereof is administered in the form of a unit-dose composition, such as an oral unit dose for the treatment and/or prevention of the disorder, such as pulmonary hypertension.

Daily amounts for PDE5 inhibitors will be in the range of 50 mg to 250 mg, which may be adjusted by increments of 5 mg, to encompass therapeutically useful ranges. For instance the range may be 50 mg to 225 mg, 50 mg to 215 mg, 50 mg to 200 mg, etc., or 55 mg to 250 mg, 60 mg to 250 mg, 65 mg to 250 mg, or 65 mg to 215 mg, 70 mg to 210 mg, etc. and variations thereof. Daily amounts for ET_{A} antagonists will be in the range of 5 mg to 125 mg, which may be adjusted by increments of 5 mg, to encompass therapeutically useful ranges. For instance the range may be 10 mg to 125 mg, 15 mg to 125 mg, 20 mg to 125 mg, etc., or 5 mg to 120 mg, 5 mg to 115 mg, 5 mg to 110 mg, or 25 mg to 90 mg, 30 mg to 85 mg etc. and variations thereof. Preferably the range of PDE5 inhibitor to ET_{A} antagonist in a ratio of 5:1 to 2:1.

Formulation of Pharmaceutical Compositions

The administration of any compound of this invention may be by any suitable means that results in a concentration of the compound that is effective for the treatment. The compound(s) may be contained in any appropriate amount in any suitable carrier substance, and is generally present in an amount of 1-95% by weight of the total weight of the composition. The composition may be provided in a dosage form that is suitable for the oral route. Thus, the composition(s) may be in the form of, e.g., tablets, capsules, pills, powders, granulates, suspensions, emulsions, solution or gels. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound (drug) substantially immediately upon administration or at any predetermined time or time period after administration. The latter types of compositions are generally known as controlled release formulations, which include (i) formulations that create a substantially constant concentration of the drug within the body over an extended period of time; (ii) formulations that, after a predetermined lag time, create a substantially constant concentration of the drug within the body over an extended period of time; (iii) formulations that sustain drug action during a predetermined time period by maintaining a relatively constant, effective drug level in the body with concomitant minimization of undesirable side effects associated with fluctuations in the plasma level of the active drug substance; (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type. Controlled release formulations may also release at least two active ingredients at different rates. In addition, controlled release formulations may release at least one active ingredient over various lengths of time, such as 12 hours or 24 hours. In one embodiment of the invention, the controlled release formulation releases at least one active ingredient over 24 hours.

Other embodiments of the invention include a pharmaceutical composition comprising an immediate release formulation and a controlled release formulation, wherein the immediate release formulation comprises an ET_{A} antagonist, a PDE5 inhibitor or both and the controlled release formulation comprises an ET_{A} antagonist, a PDE5 inhibitor or both. In one embodiment of the invention, the immediate release formulation comprises sitaxsentan and the controlled release formulation comprises sildenafil.

Administration of compounds in the form of a controlled release formulation is especially preferred in cases in which the compounds in combination, have (i) a narrow therapeutic index (i.e., the difference between the plasma concentration leading to harmful side effects or toxic reactions and the plasma concentration leading to a therapeutic effect is small); (ii) a narrow absorption window in the gastrointestinal tract; or (iii) a very short biological half-life so that frequent dosing during a day is required in order to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the drug is formulated with appropriate excipients in a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes.

Solid Dosage Forms For Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern (e.g., in order to achieve a controlled release formulation) or it may be adapted not to release the active drug substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active drug substance). The coating may be applied on the solid dosage form in a similar manner as that described in Encyclopedia of Pharmaceutical Technology, supra.

If more than one drug is administered simultaneously, the drugs may be mixed together in the tablet, or may be partitioned. In one example, a first drug is contained on the inside of the tablet, and a second drug is on the outside, such that a substantial portion of the second drug is released prior to the release of the first drug.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active drug by controlling the dissolution and/or the diffusion of the active drug substance. Controlled release formulations may also release at least two active ingredients at different rates.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration

Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents. Oral formulations also include conventional sustained release formulations, such as tablets or granules having an enteric coating.

Compositions for use in the treatment and/or prevention of pulmonary hypertension may be presented for administration to the respiratory tract as a snuff or an aerosol or solution for a nebulizer, or as a microfine powder for insufflation, alone or in combination with an inert carrier such as lactose. In such a case, the particles of active compound suitably have diameters of less than 50 microns, preferably less than 10 microns, for example between 1 and 5 microns, such as between 2 and 5 microns. A favored inhaled dose will be in the range of about 0.05 mg to 2 mg, for example about 0.05 mg to 0.5 mg, about 0.1 mg to 1 mg or about 0.5 mg to 2 mg.

As is common practice, the compositions will usually be accompanied by written or printed directions for use in the medical treatment concerned.

### EXAMPLES

The following examples present illustrative, but non-limiting, embodiments of the present disclosure.

### Example 1

### Pharmacokinetic Drug Interaction Study

A pharmacokinetic drug interaction study was performed using 24 individuals. In the study, a group of 24 normal, healthy volunteers participated in two treatment periods. During one treatment period, the subjects received 100 mg of sitaxsentan sodium (THELIN) for seven days and a single dose of 100 mg sildenafil (VIAGRA) on the last day. During the other treatment period, the subjects received placebo for seven days and 100 mg of VIAGRA on the seventh day. Twelve subjects were randomly assigned to each treatment period. When the subjects finished one treatment period, they were switched to the other treatment period. Two subjects (one in each group) did not complete the study.

Each subject's blood was drawn, using EDTA as the anticoagulant, to determine plasma levels of sitaxsentan, sildenafil and N-desmethyl sildenafil. Samples were drawn (in duplicate) once on days 1, 3, 5 and 6; 15 times on day 7 and once again on day 8 for each treatment period. The samples were stored at a nominal temperature of −20° C. for a duration not exceeding 38 days. All samples were analyzed with a set of calibration standards and low, medium and high concentration Quality Control samples.

Results showed the sildenafil administration did not alter sitaxsentan levels. Table 5 summarizes the pharmacokinetic parameters for sildenafil and N-desmethyl sildenafil after oral administration of a single 100 mg dose of sildenafil to healthy subjects after 100 mg of sitaxsentan or placebo daily for seven days in the presence of sitaxsentan The Cₘₐₓ (maximum concentration) of sildenafil increased by 18% and the AUC_{∞} (area under the plasma concentration/time curve) increased by 28%. No effects on levels of the active metabolite, N-desmethyl sildenafil, were observed. Table 6 shows a statistical comparison of pharmacokinetic parameters for sildenafil and N-desmethyl sildenafil after oral administration of a single 100 mg dose of sildenafil to healthy subjects after 100 mg of sitaxsentan or placebo daily for seven days.

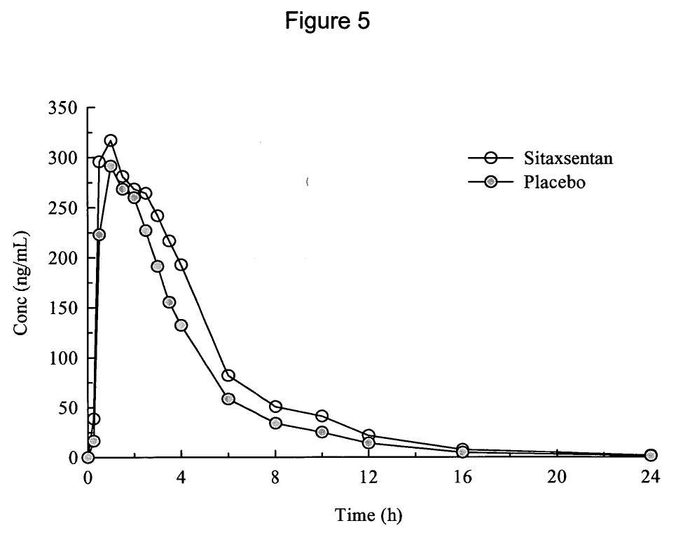

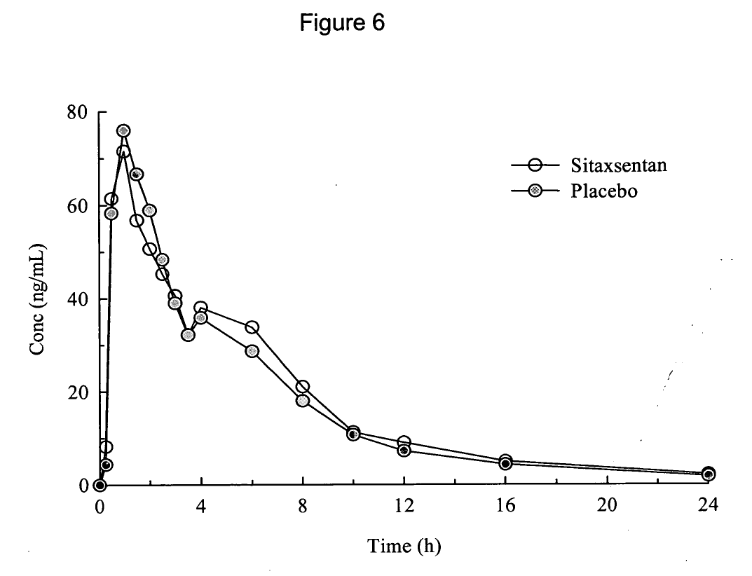

The mean plasma concentrations of sildenafil after oral administration of a single 100 mg doses of sildenafil to healthy subjects after a 100 mg dose of sitaxsentan or placebo daily for seven days are depicted in FIG. 5, and the mean plasma concentrations of N-desmethyl sildenafil after oral administration of a single 100 mg dose of sildenafil to healthy subjects after a 100 mg dose of sitaxsentan or placebo daily for seven days are depicted in FIG. 6.

Based on these data, VIAGRA does not appear to impact THELIN pharmacokinetics. THELIN showed a minor effect on overall VIAGRA pharmacokenetics, presumably based on the expected, weak, cytochrome P450 3A4 inhibition seen in cultured hepatocytes.

TABLE 5
Summary of pharmacokinetic parameters for
sildenafil and N-desmethyl sildenafil after oral
administration of a single 100 mg dose of
sildenafil to healthy subjects after 100 mg of
sitaxsentan or placebo daily for seven days.

| | | |
|---|---|---|
| Parameter¹ | Sitaxsentan | Placebo |

Sildenafil

| | | |
|---|---|---|
| Cmax (ng/mL) | 440 ± 232 | 377 ± 208 |
| Tmax (h) | 1.25 | 1.00 |
| AUC₀₋ₜ (h · ng/mL) | 1,575 ± 671 | 1,222 ± 474 |
| AUC_{∞} (h · ng/mL) | 1,648 ± 692 | 1,295 ± 488 |
| λz (h⁻¹) | 0.2514 ± 0.0364 | 0.2584 ± 0.0444 |
| t½ (h) | 2.82 ± 0.45 | 2.76 ± 0.45 |
| CL/F (mL/min) | 147 ± 56.4 | 184 ± 69.0 |
| Vz/F (L) | 34.8 ± 11.5 | 43.6 ± 18.9 |
| N-Desmethyl Sildenafil | | |
| Cmax (ng/mL) | 104 ± 40.8 | 109 ± 53.1 |
| Tmax (h) | 1.00 | 1.00 |
| AUC₀₋ₜ (h · ng/mL) | 389 ± 105 | 372 ± 107 |
| AUC_{∞} (h · ng/mL) | 425 ± 115 | 440 ± 112 |
| λz (h⁻¹) | 0.1544 ± 0.0552 | 0.1506 ± 0.0569 |
| t½ (h) | 5.14 ± 2.13 | 05.1 ± 1.63 |
| CL/F (mL/min) | 529 ± 164 | 524 ± 234 |
| Vz/F (L) | 224 ± 76.4 | 217 ± 66.2 |

¹Mean ± standard deviation except for Tmax, for which the median is reported.

TABLE 6
Statistical comparison of pharmacokinetic
parameters for sildenafil and N-desmethyl
sildenafil after oral administration of a single 100 mg
dose of sildenafil to healthy subjects after 100 mg
of sitaxsentan or placebo daily for seven days.

Ratio (%)¹

| | | |
|---|---|---|
| Parameter | Estimate | 90% Confidence Interval |

Sildenafil

| | | |
|---|---|---|
| Cmax | 117.61 | 93.81 → 147.46 |
| AUC₀₋ₜ | 124.95 | 113.40 → 137.68 |
| AUC_{∞} | 127.69 | 115.46 → 141.22 |
| CL/F | 80.03 | 72.63 → 88.18 |
| Vz/F | 81.50 | 70.98 → 93.59 |
| N-Desmethyl Sildenafil | | |
| Cmax | 100.70 | 82.72 → 122.60 |
| AUC₀₋ₜ | 112.43 | 98.50 → 128.32 |
| AUC_{∞} | 105.78 | 96.21 → 116.30 |
| CL/F | 88.95 | 77.93 → 101.53 |
| Vz/F | 94.89 | 75.60 → 119.10 |

¹GeomETic mean ratio. Based on analysis of natural log-transformed data.

### Example 2

### Efficacy Study

A randomized, double-blind, placebo-controlled study is performed comprising 60 subjects who have moderate to severe pulmonary arterial hypertension resulting form one f the following conditions: idiopathic pulmonary arterial hypertension (PAH, also known as primary pulmonary hypertension); connective tissue disease (CTD); or congenital heart disease (CHD). The subjects are randomly assigned to one of the following dosage regimes (12 subjects per treatment):

(i) placebo and placebo;

(ii) placebo and 25 mg of THELIN;

(iii) placebo and 60 mg of sildenafil;

(iv) 25 mg of THELIN and 60 mg of sildenafil; and

(v) 50 mg of THELIN and 120 mg of sildenafil.

Blood samples are collected to determine the level of THELIN in plasma and to assess sildenafil and N-desmethyl sildenafil pharmacokinetics and pharmacodynamics. Additionally, 6 minute walking distance tests are conducted to evaluate the change in the subjects' exercise capacity. Finally, symptoms of PAH are evaluated using NYHA/WHP functional class and rate of clinical worsening.

Minimal drug interaction and side effects with treatment will occur in the combination THELIN and Sildenafil of groups (iv) and (v) while maintaining successful therapeutic effect.

1. A method of treating pulmonary hypertension comprising administering to a subject in need thereof an effective amount of a) a phosphodiesterase 5 (PDE5) inhibitor and b) an endothelin A receptor (ET_{A}) antagonist.

2. A method of reducing side effects or toxicity of an ET_{A} antagonist, a PDE5 inhibitor or both comprising administering a PDE5 inhibitor and an ET_{A} antagonist, wherein the amount of the PDE5 required to treat a condition is reduced or modulated.

3. A method of reducing side effects or toxicity of an ET_{A} antagonist, a PDE5 inhibitor or both comprising administering a PDE5 inhibitor and an ET_{A} antagonist, wherein the amount of the ET_{A} antagonist required to treat a condition is reduced or modulated.

4. A method of effecting or facilitating the treatment of a vascular condition in a mammal comprising administering a therapeutically effective amount of a) an ET_{A} antagonist and b) a PDE5 inhibitor.

5. A method for treating a vascular condition comprising administering to a subject in need thereof a therapeutically effective amount of a) an ET_{A} antagonist and b) a PDE5 inhibitor.

6. The method of any one of claims 1-5, wherein said ET_{A} antagonist is selected from the group consisting of from 12 m, A-127772, A-1277722, ABT-627, BE 1827, BE-18257A, BE-18257B, BE-18572A/B, BMS-182874, BMS-193884, BMS-20794, BQ-123, BQ-153, BQ-162, BQ-485, BQ-610, BQ-745, EMD-122946, EMD-94246, FR-139317, J-104121, J-104132, JKC-301, JKC-302, L-744453, L-749329, L-754142, LU127043, LU135252, LU208075, LU302146, PD-147953, PD-151242, PD-155080, PD-156707, RO 61-1790, S-0139, SB 209670, SB 217242, SB-234551, SB-247083, sitaxsentan, sulfisoxazole, TA-0115, TA-0201, TAK-044, TBC11251, TTA-386, WS-7338B, ZD1611 and mixtures thereof.

7. The method of any one of claims 1-5, wherein said phosphodiesterase 5 inhibitor is selected from the group consisting of vardenafil, tadalafil, zaprinast, dasantafil, MBCQ, MY-5445, dipyridamole, furoyl and benzofuroyl pyrroloquinolones, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, T-1032 (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, RX-RA-69, SCH-51866, KT-734, vesnarinone, zaprinast, SKF-96231, ER-21355, BF/GP-385, NM-702 and mixtures thereof.

8. The method of any one of claims 1-5, wherein the ET_{A} antagonist is selected from ABT-627, sitaxsentan, sulfisoxazole, TBC11251, ZD1611 and mixtures thereof.

9. The method of any one of claims 1-5, wherein the PDE5 inhibitor is selected from vardenafil, tadalafil, dasantafil, dipyridamole, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, vesnarinone, zaprinast, and mixtures thereof.

10. The method of any one of claims 1-5, wherein the ET_{A} antagonist is sitaxsentan

11. The method of any one of claims 1-5, wherein the PDE5 inhibitor is sildenafil.

12. The method of any one of claims 1-5, wherein the PDE5 inhibitor is tadalafil.

13. The method of any one of claims 1-5, wherein the ET_{A} antagonist is sitaxsentan and the PDE5 inhibitor is sildenafil.

14. The method of any one of claims 1-5, wherein the ET_{A} antagonist is sitaxsentan and the PDE5 inhibitor is tadalafil.

15. The method of claims 4 or 5, wherein said vascular condition is selected from the group consisting of erectile dysfunction, atherosclerosis, renal failure, hypertension, congestive heart failure, diabetic nephropathy, diabetic neuropathy, interstitial lung disease, obstructive sleep dyspnea, obstructive sleep apnea and resistant hypertension.

16. The method of claims 4 or 5, wherein said vascular condition is a cardiovascular condition.

17. The method of claim 15, wherein (a) and (b) are administered substantially concurrently.

18. The method of claim 15, wherein (a) and (b) are administered sequentially.

19. A combination therapy comprising at least one endothelin A receptor (ET_{A}) antagonist and a phosphodiesterase 5 (PDE5) inhibitor.

20. The combination therapy of claim 19, wherein said ET_{A} antagonist is selected from the group consisting of 12 m, A-127772, A-1277722, ABT-627, BE 1827, BE-18257A, BE-18257B, BE-18572A/B, BMS-182874, BMS-193884, BMS-20794, BQ-123, BQ-153, BQ-162, BQ-485, BQ-610, BQ-745, EMD-122946, EMD-94246, FR-139317, J-104121, J-104132, JKC-301, JKC-302, L-744453, L-749329, L-754142, LU127043, LU135252, LU208075, LU302146, PD-147953, PD-151242, PD-155080, PD-156707, RO 61-1790, S-0139, SB 209670, SB 217242, SB-234551, SB-247083, sitaxsentan, sulfisoxazole, TA-0115, TA-0201, TAK-044, TBC11251, TTA-386, WS-7338B, ZD1611 and mixtures thereof.

21. The combination therapy of claim 19, wherein said PDE5 inhibitor is selected from the group consisting of vardenafil, tadalafil, dasantafil, MBCQ, MY-5445, dipyridamole, furoyl and benzofuroyl pyrroloquinolones, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, T-1032 (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, RX-RA-69, SCH-51866, KT-734, vesnarinone, zaprinast, SKF-96231, ER-21355, BF/GP-385, NM-702 and mixtures thereof.

22. The combination therapy of claim 19, wherein the ET_{A} antagonist is selected from ABT-627, sitaxsentan, sulfisoxazole, TBC11251, ZD1611 and mixtures thereof.

23. The combination therapy of claim 19, wherein the PDE5 inhibitor is selected from vardenafil, tadalafil, dasantafil, dipyridamole, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, vesnarinone, zaprinast, and mixtures thereof.

24. The combination therapy of claim 19, wherein the ET_{A} antagonist is sitaxsentan

25. The combination therapy of claim 19, wherein the PDE5 inhibitor is sildenafil.

26. The combination therapy of claim 19, wherein the PDE5 inhibitor is tadalafil.

27. The combination therapy of claim 19, wherein the ET_{A} antagonist is sitaxsentan and the PDE5 inhibitor is sildenafil.

28. The combination therapy of claim 19, wherein the ET_{A} antagonist is sitaxsentan and the PDE5 inhibitor is tadalafil.

29. The combination therapy of claim 19, wherein the ET_{A} antagonist and PDE5 inhibitor are administered together or separately.

30. The combination therapy of claim 19, wherein the combination therapy is a pharmaceutical composition.

31. The combination therapy of claim 19, wherein the pharmaceutical composition is in an immediate release formulation.

32. The combination therapy of claim 30, wherein the ET_{A} antagonist is in a controlled release formulation, the PDE5 inhibitor is in a controlled release formulation, or both are in a controlled release formulation.

33. The combination therapy of claim 30, wherein both the ET_{A} antagonist and the PDE5 inhibitor are in a controlled release formulation, but the ET_{A} antagonist and the PDE5 inhibitor are released at different rates.

34. A pharmaceutical composition comprising an ET_{A} antagonist, a PDE5 inhibitor and a pharmaceutical carrier.

35. The pharmaceutical composition of claim 34, wherein said ET_{A} antagonist is selected from the group consisting of 12 m, A-127772, A-1277722, ABT-627, BE 1827, BE-18257A, BE-18257B, BE-18572A/B, BMS-182874, BMS-193884, BMS-20794, BQ-123, BQ-153, BQ-162, BQ-485, BQ-610, BQ-745, EMD-122946, EMD-94246, FR-139317, J-104121, J-104132, JKC-301, JKC-302, L-744453, L-749329, L-754142, LU127043, LU135252, LU208075, LU302146, PD-147953, PD-151242, PD-155080, PD-156707, RO 61-1790, S-0139, SB 209670, SB 217242, SB-234551, SB-247083, sitaxsentan, sulfisoxazole, TA-0115, TA-0201, TAK-044, TBC11251, TTA-386, WS-7338B, ZD1611 and mixtures thereof.

36. The pharmaceutical composition of claim 34, wherein said PDE5 inhibitor is selected from the group consisting of vardenafil, tadalafil, dasantafil, MBCQ, MY-5445, dipyridamole, furoyl and benzofuroyl pyrroloquinolones, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, T-1032 (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, RX-RA-69, SCH-51866, KT-734, vesnarinone, zaprinast, SKF-96231, ER-21355, BF/GP-385, NM-702 and mixtures thereof.

37. The pharmaceutical composition of claim 34, wherein the ET_{A} antagonist is selected from ABT-627, sitaxsentan, sulfisoxazole, TBC11251, ZD1611 and mixtures thereof.

38. The pharmaceutical composition of claim 34, wherein the PDE5 inhibitor is selected from vardenafil, tadalafil, dasantafil, dipyridamole, 2-(2-Methylpyridin-4-yl)methyl-4-(3,4,5-trimethoxyphen-yl)-8-(pyrimidin-2-yl)methoxy-1,2-dihydro-1-oxo-2,7-naphthyridine-3-carbox-ylic acid methyl ester hydrochloride, (methyl 2-(4-aminophenyl)-1,2-dihydro-1-oxo-7-(2-pyridylmethoxy)-4-(3,4,5-trimeth-oxy-phenyl)-3-isoquinoline carboxylate sulfate), sildenafil, vesnarinone, zaprinast, and mixtures thereof.

39. The pharmaceutical composition of claim 34, wherein the ET_{A} antagonist is sitaxsentan

40. The pharmaceutical composition of claim 34, wherein the PDE5 inhibitor is sildenafil.

41. The pharmaceutical composition of claim 34, wherein the PDE5 inhibitor is tadalafil.

42. The pharmaceutical composition of claim 34, wherein the ET_{A} antagonist is sitaxsentan and the PDE5 inhibitor is sildenafil.

43. The pharmaceutical composition of claim 34, wherein the ET_{A} antagonist is sitaxsentan and the PDE5 inhibitor is tadalafil.

44. The pharmaceutical composition of claim 34, wherein the pharmaceutical composition is in an immediate release formulation.

45. The pharmaceutical composition of claim 34, wherein the ET_{A} antagonist is in a controlled release formulation, the PDE5 inhibitor is in a controlled release formulation, or both are in a controlled release formulation.

46. The pharmaceutical composition of claim 34, wherein both the ET_{A} antagonist and the PDE5 inhibitor are in a controlled release formulation, but the ET_{A} antagonist and the PDE5 inhibitor are released at different rates.
